(19) United States
(12) Patent Application Publication
(10) Pub. No.: US 2009/0163546 A1
(43) Pub. Date: Jun. 25, 2009

(54) DIACYLGLYCEROL ACYLTRANSFERASE INHIBITORS

(76) Inventors: David Robert Bolin, Montclair, NJ (US); Jianping Cai, West Caldwell, NJ (US); Robert Alan Goodnow, JR., Gillette, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/325,322

(22) Filed: Dec. 1, 2008

Related U.S. Application Data
(60) Provisional application No. 61/012,073, filed on Dec. 7, 2007.

(51) Int. Cl. A61K 31/4709; C07D 231/56; C07D 401/12; A61K 31/454; A61K 31/416; A61P 3/04; A61P 3/10

(52) U.S. Cl. 514/314; 548/361.5; 546/171; 546/199; 514/322; 514/405

(57) ABSTRACT

Provided herein are compounds of the formula (I):

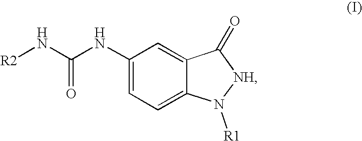

as well as pharmaceutically acceptable salts thereof, wherein the substituents are as those disclosed in the specification. These compounds, and the pharmaceutical compositions containing them, are useful for the treatment of diseases such as, for example, obesity, type II diabetes mellitus and metabolic syndrome.

### PRIORITY TO RELATED APPLICATION(S)

This application claims the benefit of U.S. Provisional Application No. 61/012,073, filed Dec. 7, 2007, which is hereby incorporated by reference in its entirety.

### FIELD OF THE INVENTION

The invention relates to inhibitors of diacylglycerol acyltransferase. The inhibitors are useful for the treatment of diseases such as obesity, type II diabetes mellitus, dyslipidemia and metabolic syndrome.

All documents cited or relied upon below are expressly incorporated herein by reference.

### BACKGROUND OF THE INVENTION

Triglycerides or triacylglycerols are the major form of energy storage in eukaryotic organisms. In mammals, these compounds are primarily synthesized in three tissues: the small intestine, liver, and adipocytes. Triglycerides or triacylglycerols support the major functions of dietary fat absorption, packaging of newly synthesized fatty acids and storage in fat tissue (see Subauste and Burant, Current Drug Targets—Immune, Endocrine & Metabolic Disorders (2003) 3, 263-270).

Diacylglycerol O-acyltransferase, also known as diglyceride acyltransferase or DGAT, is a key enzyme in triglyceride synthesis. DGAT catalyzes the final and rate-limiting step in triacylglycerol synthesis from 1,2-diacylglycerol (DAG) and long chain fatty acyl CoA as substrates. Thus, DGAT plays an essential role in the metabolism of cellular diacylglycerol and is critically important for triglyceride production and energy storage homeostasis (see Mayorek et al, European Journal of Biochemistry (1989) 182, 395-400).

DGAT has a specificity for sn-1,2 diacylglycerols and will accept a wide variety of fatty acyl chain lengths (see Farese et al, Current Opinions in Lipidology (2000) 11, 229-234). DGAT activity levels increase in fat cells as they differentiate in vitro and recent evidence suggests that DGAT may be regulated in adipose tissue post-transcriptionally (see Coleman et al, Journal of Molecular Biology (1978) 253, 7256-7261 and Yu et al, Journal of Molecular Biology (2002) 277, 50876-50884). DGAT activity is primarily expressed in the endoplasmic reticulum (see Colman, Methods in Enzymology (1992) 209, 98-104). In hepatocytes, DGAT activity has been shown to be expressed on both the cytosolic and luminal surfaces of the endoplasmic reticular membrane (see Owen et al, Biochemical Journal (1997) 323 (pt 1), 17-21 and Waterman et al, Journal of Lipid Research (2002) 43, 1555-156). In the liver, the regulation of triglyceride synthesis and partitioning, between retention as cytosolic droplets and secretion, is of primary importance in determining the rate of VLDL production (see Shelness and Sellers, Current Opinions in Lipidology (2001) 12, 151-157 and Owen et al, Biochemical Journal (1997) 323 (pt 1), 17-21).

Two forms of DGAT have been cloned and are designated DGAT1 and DGAT2 (see Cases et al, Proceedings of the National Academy of Science, USA (1998) 95, 13018-13023, Lardizabal et al, Journal of Biological Chemistry (2001) 276, 38862-38869 and Cases et al, Journal of Biological Chemistry (2001) 276, 38870-38876). Although both enzymes utilize the same substrates, there is no homology between DGAT1 and DGAT2. Both enzymes are widely expressed however some differences do exist in the relative abundance of expression in various tissues.

The gene encoding mouse DGAT1 has been used to create DGAT knock-out. These mice, although unable to express a functional DGAT enzyme (Dgat-/- mice), are viable and continue to synthesize triglycerides (see Smith et al, Nature Genetics (2000) 25, 87-90). This would suggest that multiple catalytic mechanisms contribute to triglyceride synthesis, such as DGAT2. An alternative pathway has also been shown to form triglycerides from two diacylglycerols by the action of diacylglycerol transacylase (see Lehner and Kuksis, Progress in Lipid Research (1996) 35, 169-210).

Dgat-/- mice are resistant to diet-induced obesity and remain lean. When fed a high fat diet, Dgat-/- mice maintain weights comparable to mice fed a diet with regular fat content. Dgat-/- mice have lower tissue triglyceride levels. The resistance to weight gain seen in the knockout mice, which have a slightly higher food intake, is due to an increased energy expenditure and increased sensitivity to insulin and leptin (see Smith et al, Nature Genetics (2000) 25, 87-90, Chen and Farese, Trends in Cardiovascular Medicine (2000) 10, 188-192, Chen and Farese, Current Opinions in Clinical Nutrition and Metabolic Care (2002) 5, 359-363 and Chen et al, Journal of Clinical Investigation (2002) 109, 1049-1055). Dgat-/- mice have reduced rates of triglyceride absorption, improved triglyceride metabolism, and improved glucose metabolism, with lower glucose and insulin levels following a glucose load, in comparison to wild-type mice (see Buhman et al, Journal of Biological Chemistry (2002) 277, 25474-25479 and Chen and Farese, Trends in Cardiovascular Medicine (2000) 10, 188-192).

Disorders or imbalances in triglyceride metabolism, both absorption as well as de novo synthesis, have been implicated in the pathogenesis of a variety of disease risks These include obesity, insulin resistance syndrome, type II diabetes, dyslipidemia, metabolic syndrome (syndrome X) and coronary heart disease (see Kahn, Nature Genetics (2000) 25, 6-7, Yanovski and Yanovski, New England Journal of Medicine (2002) 346, 591-602, Lewis et al, Endocrine Reviews (2002) 23, 201, Brazil, Nature Reviews Drug Discovery (2002) 1, 408, Malloy and Kane, Advances in Internal Medicine (2001) 47, 111, Subauste and Burant, Current Drug Targets—Immune, Endocrine & Metabolic Disorders (2003) 3, 263-270 and Yu and Ginsberg, Annals of Medicine (2004) 36, 252-261). Compounds that can decrease the synthesis of triglycerides from diacylglycerol by inhibiting or lowering the activity of the DGAT enzyme would be of value as therapeutic agents for the treatment diseases associated with abnormal metabolism of triglycerides.

Known inhibitors of DGAT include: dibenzoxazepinones (see Ramharack, et al, EP1219716 and Burrows et al, 26^{th} National Medicinal Chemistry Symposium (1998) poster C-22), substituted amino-pyrimidino-oxazines (see Fox et al, WO2004047755), chalcones such as xanthohumol (see Tabata et al, Phytochemistry (1997) 46, 683-687 and Casaschi et al, Journal of Nutrition (2004) 134, 1340-1346), substituted benzyl-phosphonates (see Kurogi et al, Journal of Medicinal Chemistry (1996) 39, 14331-1437, Goto, et al, Chemistry and Pharmaceutical Bulletin (1996) 44, 547-551, Ikeda, et al, Thirteenth International Symposium on Athersclerosis (2003), abstract 2P-0401, and Miyata, et al, JP 2004067635), aryl alkyl acid derivatives (see Smith et al, WO2004100881 and US20040224997), furan and thiophene derivatives (see WO2004022551), pyrrolo[1,2b]pyridazine derivatives (see Fox et al, WO2005103907), and substituted sulfonamides (see Budd Haeberlein and Buckett, WO20050442500).

Also known to be inhibitors of DGAT are: 2-bromo-palmitic acid (see Colman et al, Biochimica et Biophysica Acta (1992) 1125, 203-9), 2-bromno-octanoic acid (see Mayorek and Bar-Tana, Journal of Biological Chemistry (1985) 260, 6528-6532), roselipins (see Noriko et al, (Journal of Antibiotics (1999) 52, 815-826), amidepsin (see Tomoda et al, Journal of Antibiotics (1995) 48, 942-7), isochromophilone, prenylflavonoids (see Chung et al, Planta Medica (2004) 70, 258-260), polyacetylenes (see Lee et al, Planta Medica (2004) 70, 197-200), cochlioquinones (see Lee et al, Journal of Antibiotics (2003) 56, 967-969), tanshinones (see Ko et al, Archives of Pharmaceutical Research (2002) 25, 446-448), gemfibrozil (see Zhu et al, Atherosclerosis (2002) 164, 221-228), and substituted quinolones (see Ko, et al, Planta Medica (2002) 68, 1131-1133). Also known to be modulators of DGAT activity are antisense oligonucleotides (see Monia and Graham, US200401 85559).

A need exits in the art, however, for additional DGAT inhibitors that have efficacy for the treatment of metabolic disorders such as, for example, obesity, type II diabetes mellitus and metabolic syndrome. Further, a need exists in the art for DGAT inhibitors having IC₅₀ values less than about 1 μM.

### SUMMARY OF THE INVENTION

The present invention pertains to DGAT inhibitors In a preferred embodiment, the invention provides compounds of the formula (I):

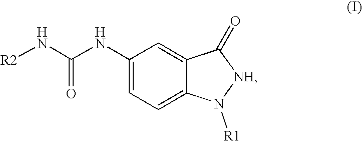

as well as pharmaceutically acceptable salts thereof.

In another embodiment of the present invention, provided is a pharmaceutical composition, comprising a therapeutically effective amount of a compound according to formula I or a pharmaceutically acceptable salt thereof and a pharmaceutically acceptable carrier.

### DETAILED DESCRIPTION OF THE INVENTION

In a preferred embodiment of the present invention, provided are compounds of formula I:

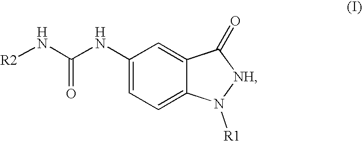

wherein:

R¹ is
—CH₂-aryl,
-lower alkyl,
—CH₂CH(O)OCH₂CH₃,
—CH₂CH₂OCH₂CH₂OCH₃, or
—CH₂CH₂OCH₃; and

R² is
-aryl, unsubstituted or mono- or bisubstituted with halogen, lower alkyl, alkoxy, O-haloalkyl, haloalkyl, heterocycloalkyl or ethynyl moiety, or
-bicyclic heteroaryl;
   and pharmaceutically acceptable salts thereof.

In another preferred embodiment of the present invention, provided is a pharmaceutical composition, comprising a therapeutically effective amount of a compound according to formula (I) or a pharmaceutically acceptable salt thereof and a pharmaceutically acceptable carrier.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “cycloalkyl” refers to a monovalent carbocyclic radical of three to seven, preferably three to six carbon atoms. This term is further exemplified by radicals such as cyclopropyl, cyclobutyl, cyclopentyl and cyclohexyl. In a preferred embodiment, the “cycloalkyl” moieties can optionally be substituted with one, two, three or four substituents, wherein each substituent is independently, for example, hydroxy, alkyl, alkoxy, halogen or amino, unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a cyclic alkyl ring, wherein one, two or three of the carbon ring atoms is replaced by a heteroatom such as N, O or S. Examples of heterocycloalkyl groups include, but are not limited to, morpholine, thiomorpholine, piperazine, piperidine and the like. The heterocycloalkyl groups may be unsubstituted or substituted.

The term “lower alkyl”, alone or in combination with other groups, refers to a branched or straight-chain monovalent alkyl radical of one to six carbon atoms, preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic monovalent mono- or polycarbocyclic radical, such as phenyl or naphthyl, preferably phenyl.

The term “heteroaryl,” alone or in combination with other groups, means a monocyclic or bicyclic radical of 5 to 12 ring atoms having at least one aromatic ring containing one, two, or three ring heteroatoms selected from N, O, and S, the remaining ring atoms being C. A preferred bicyclic heteroaryl is quinoline. One or two ring carbon atoms of the heteroaryl group may be replaced with a carbonyl group. The heteroaryl group described above may be substituted independently with one, two, or three substituents, preferably one or two substituents such as, for example, halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, amino C₁₋₆ alkyl, mono- or di-substituted amino-C₁₋₆ alkyl, nitro, cyano, acyl, carbamoyl, mono- or di-substituted amino, aminocarbonyl, mono- or di-substituted amino-carbonyl, aminocarbonyl C₁₋₆ alkoxy, mono- or di-substituted amino-carbonyl-C₁₋₆ alkoxy, hydroxy-C₁₋₆ alkyl, carboxyl, C₁₋₆ alkoxy carbonyl, aryl C₁₋₆ alkoxy, heteroaryl C₁₋₆ alkoxy, heterocyclyl C₁₋₆ alkoxy, C₁₋₆ alkoxycarbonyl C₁₋₆ alkoxy, carbamoyl C₁₋₆ alkoxy and carboxyl C₁₋₆ alkoxy, preferably halogen, hydroxy, C₁₋₆ alkyl, halo C₁₋₆ alkyl, C₁₋₆ alkoxy, C₁₋₆ alkyl sulfonyl, C₁₋₆ alkyl sulfinyl, C₁₋₆ alkylthio, amino, mono-C₁₋₆ alkyl substituted amino, di-C₁₋₆ alkyl substituted amino, amino C₁₋₆ alkyl, mono-C₁₋₆ alkyl substituted amino-C₁₋₆ alkyl, di-C₁₋₆ alkyl substituted amino-C₁₋₆ alkyl, nitro, carbamoyl, mono- or di-substituted amino-carbonyl, hydroxy-C₁₋₆ alkyl, carboxyl, C₁₋₆ alkoxy carbonyl and cyano.

The alkyl and aryl groups may be substituted or unsubstituted. Where substituted, there will generally be, for example, 1 to 3 substituents present, preferably 1 substituent. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono-or di-alkyl aminocarbonylalkyl, arylaminocarbonyl), carbamates (e.g. alkoxycarbonylamino, arloxycarbonylamino, aminocarbonyloxy, mono-or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more, preferably one, heteroatom, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridaziniyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

The lower alkyl groups may be substituted or unsubstituted, preferably unsubstituted. Where substituted, there will generally be, for example, 1 to 3 substitutents present, preferably 1 substituent.

As used herein, the term “alkoxy” means alkyl-O—; and “alkoyl” means alkyl-CO—. Alkoxy substituent groups or alkoxy-containing substituent groups may be substituted by, for example, one or more alkyl groups.

As used herein, the term “halogen” means a fluorine, chlorine, bromine or iodine radical, preferably a fluorine, chlorine or bromine radical, and more preferably a fluorine or chlorine radical.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucinc, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminium salts.

In the practice of the method of the present invention, an effective amount of ally one of the compounds of this invention or a combination of any of the compounds of this invention or a pharmaceutically acceptable salt thereof is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition call also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by F. W. Martin. Such compositions will, in ally event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day

Compounds of the present invention can be prepared beginning with commercially available starting materials and utilizing general synthetic techniques and procedures known to those skilled in the art. Outlined below are reaction schemes suitable for preparing such compounds. Further exemplification is found in the specific examples listed below.

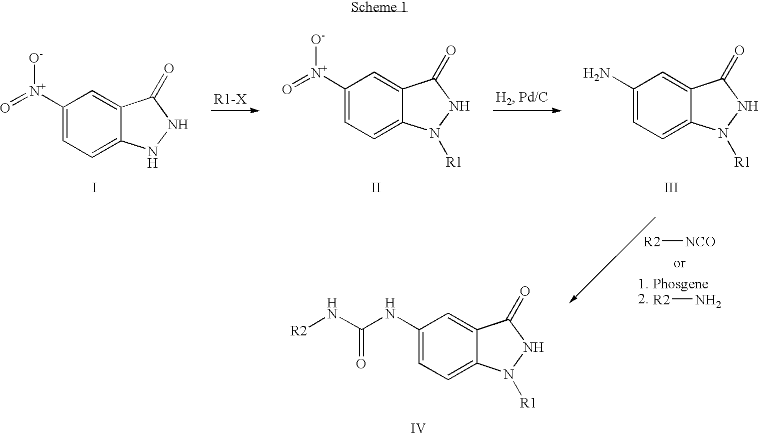

As shown in Scheme 1, 5-nitro-1,2-dihydro-indazol-3-one (I), may be alkylated with an alkylating agent R1-X (where X is a halogen, and R1 is alkyl, arylalkyl, alkenyl or alkyloxyalkyl) in the presence of an organic or inorganic base to give indazolones II under conditions analogous to the ones described by Amrein et. al. in US 2006/0069269 A1 and Aran et. al. in Heterocycles 1997, 45, 129.

The reduction of aryl nitro compounds II to amines III is typically done in a suitable solvent using hydrogen in the presence of palladium on carbon. Amines of the general structure III call be converted to desirable ureas of the general formula IV upon treatment with a suitable isocyanate R2-NCO. Alternatively amines III can be converted to desirable ureas of the general structure IV by a reaction with phosgene followed by a reaction with a desirable amine R2-NH₂ (where R2 may be alkyl or aryl but preferred are substituted aryl).

### EXAMPLES

### List of Abbreviations/Definitions

DGAT is diacylglycerol:acyl CoA O-acyltransferase
THF is tetrahydrofuran
DMF is dimethylformamide
DMA is N,N-dimethylacetamide
DMSO is dimethylsulfoxide
DCM is dichloromethane
DME is dimethoxyethane
MeOH is methanol
EtOH is ethanol
NBS is N-Bromosuccinimide
TFA is 1,1,1-trifluoroacetic acid
HOBT is 1-hydroxybenzotriazole
PyBroP is bromotripyrrolidinophosphonium hexafluorophosphate
EDCI is 1-[3-(dimethylamino)propyl]-3ethylcarbodiimide hydrochloride
DIPEA is diisopropylethylamine
Brine is saturated aqueous solution of sodium chloride
DAG is 1,2-dioleoyl-sn-glycerol
TLC is thin layer chromatography
RP HPLC is reversed phase high performance liquid chromatography
HRMS is high resolution mass spectrometry
APCI-MS is atmospheric pressure chemical ionization mass spectrometry
ES-MS is electrospray mass spectrometry
LCMS is liquid chromatography mass spectrometry
RT is room or ambient temperature.

### Part 1: Preparation of Preferred Intermediates

### Preparation of 1-benzyl-5-nitro-1,2-dihydro-indazol-3-one

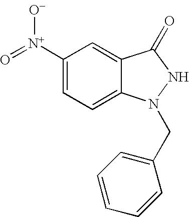

Benzyl bromide (10.5 g, 61 mmol) was added dropwise to a mixture of 5-nitro-1,2-dihydro-indazol-3-one (prepared according to Org. Synth. 1949, 29, 54 or Chem. Ber. 1942, 75, 1104) (10 g, 55.8 mmol) and NaOH (15%, 45 ml). The mixture was stirred at 80° C. for 1 h then cooled, neutralized with HCl (6N aqueous) and filtered. The solid obtained washed with water and dried in airflow. The solid was then stirred in MeOH (25 ml) and ethyl acetate (25 ml) for 1 h then filtered and washed again with MeOH-ethyl acetate. After drying the solid was suspended in water (100 ml), NaOH (15%, 10 ml) was added and the mixture was stirred for 30 min. Filtered and washed with water, the filtrate (mother liquid) was neutralized with HCl (1N aqueous) to pH=4-5. The product of 1-benzyl-5-nitro-1,2-dihydro-indazol-3-one was obtained by filtration (9.18 g, 61% yield). ES-MS calcd for C14H11N3O3 (m/e) 269, obsd 270 (M+H).

### Preparation of 1-allyl-5-nitro-1,2-dihydro-indazol-3-one

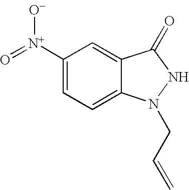

Starting from 5-nitro-1,2-dihydro-indazol-3-one and allyl bromide, 1-allyl-5-nitro-1,2-dihydro-indazol-3-one was prepared using a method similar to the one described in the synthesis of 1-benzyl-5-nitro-1,2-dihydro-indazol-3-one (67% yield). ES-MS calcd for C10H9N3O3 (m/e) 219, obsd 220 (M+H).

### Preparation of (5-nitro-3-oxo-2,3-dihydro-indazol-1-yl)-acetic acid ethyl ester

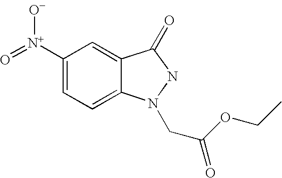

A mixture of 5-nitro-1,2-dihydro-indazol-3-one (0.5 g, 2.79 mmol), ethyl bromoacetate (309 μL, 2.79 mmol) and potassium carbonate (771 mg, 558 mmol) in DMF (5 mL) was stirred at room temperature overnight. The red reaction mixture was poured into 75 mL H₂O and 50 mL ethyl acetate. The pH of the solution was adjusted to 2 with HCl (conc) and the organic layer was separated. The aqueous layer was extracted with ethyl acetate. The combined organic layer was dried over MgSO₄, filtered and evaporated. The residue was purified by flash chromatography to afford the product (5-nitro-3-oxo-2,3-dihydro-indazol-1-yl)-acetic acid ethyl ester (0.38 g, 51%). ES-MS calcd for C11H11N3O5 (m/e) 265.22, obsd 264.1 (M−H),

### Preparation of 1-[2-(2-methoxy-ethoxy)-ethyl]-5-nitro-1,2-dihydro-indazol-3-one

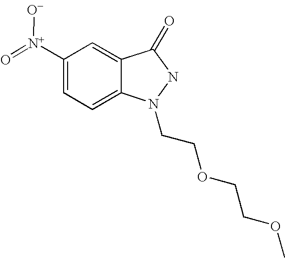

A mixture of 5-nitro-1,2-dihydro-indazol-3-one (399 mg, 2.22 mmol), 1-bromo-2-(2-methoxy-ethoxy)-ethane (454 μL, 3.34 mmol), potassium iodide (370 mg, 2.22 mmol) and 1N sodium hydroxide solution (6.7 mL, 6.7 mmol) in 2 ml dioxane was stirred at 60° C. overnight. The reaction mixture was then cooled, poured into 50 mL H₂O and 300 □L 10N NaOH was added. The aqueous layer was extracted with CH₂Cl₂ and then acidified to ˜pH 2 with 6N aqueous HCl. The aqueous layer was extracted with ethyl acetate. The combined organic layer dried over MgSO₄, filtered and evaporated. The residue was purified by flash chromatography to afford the product 1-[2-(2-methoxy-ethoxy)-ethyl]-5-nitro-1,2-dihydro-indazol-3-one (380 mg, 61%). ES-MS calcd for C12H15N3O5 (m/e) 281.26, obsd 282.17 (M+H).

### Preparation of 1-(2-methoxy-ethyl)-5-nitro-1,2-dihydro-indazol-3-one

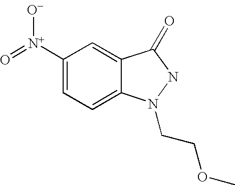

A mixture of 5-nitro-1,2-dihydro-indazol-3-one (402 mg, 2.24 mmol), 1-bromo-2-methoxy-ethane (332 μL, 3.53 mmol), potassium iodide (372 mg, 2.24 mmol) and 1N aqueous sodium hydroxide solution (6.7 mL, 6.7 mmol) in 2 ml dioxane was stirred at 60° C. for 12 hrs and cooled. The reaction mixture was poured into 50 mL H₂O and 200 μL 10N NaOH was added. The aqueous layer was extracted with ether (30 mL) and CH₂Cl₂ (3×30 mL) and then acidified with 6N aqueous HCl. The aqueous layer was extracted with ethyl acetate (6×30 mL). The organic layers were combined, dried over MgSO₄, filtered and evaporated under vacuum to a yellow solid (430 mg). The crude product was purified by flash chromatography using a AcOH/MeOH/CHCl₃ solvent system to yield 1-(2-methoxy-ethyl)-5-nitro-1,2-dihydro-indazol-3-one as a yellow solid (340 mg, Yield: 64%). ES-MS calcd for C10H11N3O4 (m/e) 237.21, obsd 238.0 (M+H).

### Part II: Preparation of Preferred Compounds of the Invention

### General method for the preparation of 1-benzyl and 1-propyl indazolone ureas from isocyanates (General method 1)

A suspension of 1-benzyl-5-nitro-1,2-dihydro-indazol-3-one (1 eq.) or 1-allyl-5-nitro-1,2-dihydro-indazol-3-one and 10% Pd/C (3-5% equiv.) in MeOH (25 ml per 1 mmol of substrate) was stirred under hydrogen atmosphere (balloon) at room temperature until completion of reduction. After removal of the catalyst and the solvent, the residue was dissolved in acetonitrile (5-15 ml) and the solution was evaporated again. The intermediate reduction product was dried in high vacuum then dissolved in a solvent (DMF or acetonitrile) to make a certain concentration of solution (0.1 to 0.25 based on the solubility). The solution (0.075 mmol) was dispensed to vials followed by adding a desirable isocyanate (0.25 M, 1 equiv.). Then the vials were shaken at 80-90° C. for 4-5 hrs. Solvent removal followed by HPLC purification offered the pure compounds.

### Example 1

### 1-(1-Benzyl-3-oxo-2,3-dihydro-1H-indazol-5-yl)-3-(5-chloro-2-methoxy-phenyl)-urea

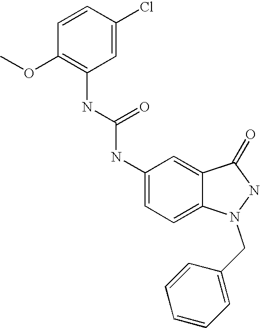

Following general method 1, described above, 1-(1-benzyl-3-oxo-2,3-dihydro-1H-indazol-5-yl)-3-(5-chloro-2-methoxy-phenyl)-urea was prepared from 1-benzyl-5-nitro-1,2-dihydro-indazol-3-one and 5-chloro-2-methoxyphenyl isocyanate (Yield: 13%). ES-MS calcd for C22H19ClN4O3 (m/e) 422, obsd 423 (M+H).

### Example 2

### 1-(1-Benzyl-3-oxo-2,3-dihydro-1H-indazol-5-yl)-3-(2-ethoxy-phenyl)-urea

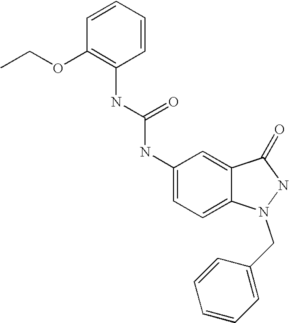

Following general method 1, described above, 1-(1-benzyl-3-oxo-2,3-dihydro-1H-indazol-5-yl)-3-(2-ethoxy-phenyl)-urea was prepared from 1-benzyl-5-nitro-1,2-dihydro-indazol-3-one and 2 ethoxyphenyl isocyanate (Yield: 48%), ES-MS calcd C23H22N4O3 for 402 (m/e), obsd 403 (M+H).

### Example 3

### 1-(1-Benzyl-3-oxo-2,3-dihydro-1H-indazol-5-yl)-3-(2-methoxy-phenyl)-urea

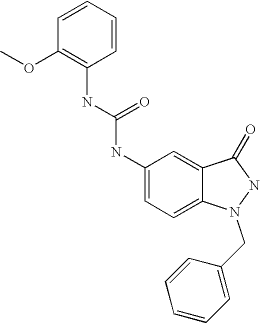

Following the general method 1, described above, 1-(1-benzyl-3-oxo-2,3-dihydro-1H-indazol-5-yl)-3-(2-methoxy-phenyl)-urea was prepared from 1-benzyl-5-nitro-1,2-dihydro-indazol-3-one and 2-methoxyphenyl isocyanate (Yield: 48%). ES-MS calcd C22H20N4Ofor 388 (m/e), obsd 389 (M+H).

### Example 4

### 1-(3-Oxo-1-propyl-2,3-dihydro-1H-indazol-5-yl)-3-(2-trifluoromethoxy-phenyl)-urea

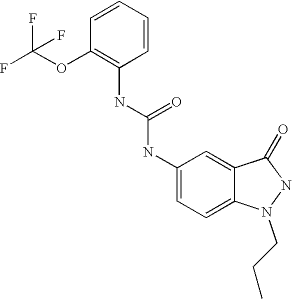

Following the general method 1, described above, 1-(3-oxo-1-propyl-2,3-dihydro-1H-indazol-5-yl)-3-(2-trifluoromethoxy-phenyl)-urea was prepared from 1-allyl-5-nitro-1,2-dihydro-indazol-3-one and 2-(trifluoromethoxy)phenyl isocyanate (Yield, 75%). ES-MS calcd for C18H17F3N4O3 (m/e) 394, obsd 395 (M+H).

### Example 5

### 1-(4-Butyl-phenyl)-3-(3-oxo-1-propyl-2,3-dihydro-1H-indazol-5-yl)-urea

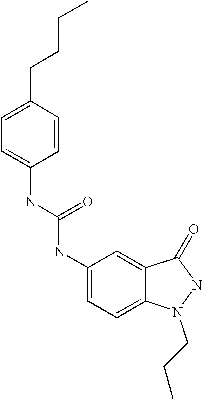

Following the general method 1, described above, 1-(4-butyl-phenyl)-3-(3-oxo-1-propyl-2,3-dihydro-1H-indazol-5-yl)-urea was prepared from 1-allyl-5-nitro-1,2-dihydro-indazol-3-one and 4-butylphenyl isocyanate (Yield: 86%). ES-MS calcd for C21H26N4O2 (m/e) 366, obsd 367 (M+H).

### Example 6

### 1-(2-Methoxy-5-methyl-phenyl)-3-(3-oxo-1-propyl-2,3-dihydro-1H-indazol-5-yl)-urea

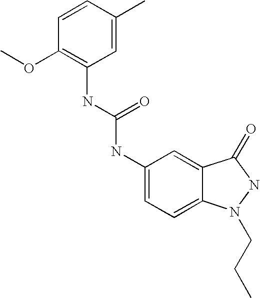

Following the general method 1, described above, 1-(2-ethoxy-5-methyl-phenyl)-3-(3-oxo-1-propyl-2,3-dihydro-1H-indazol-5-yl)-urea was prepared from 1-allyl-5-nitro-1,2-dihydro-indazol-3-one and 2-methoxy-5-methylphenyl isocyanate (Yield: 71%). ES-MS calcd for C19H22N4O3 (m/e) 354, obsd 355 (M+H).

### Example 7

### 1-(5-Chloro-2-methoxy-phenyl)-3-(3-oxo-1-propyl-2,3-dihydro-1H-indazol-5-yl)-urea

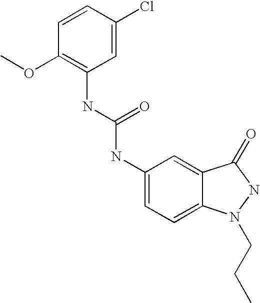

Following the general method 1, described above, 1-(5-chloro-2-methoxy-phenyl)-3-(3-oxo-1-propyl-2,3-dihydro-1H-indazol-5-yl)-urea was prepared from 1-allyl-5-nitro-1,2-dihydro-indazol-3-one and 5-chloro-2-methoxyphenyl isocyanate (Yield: 79%). ES-MS calcd for C18H19ClN4O3 (m/e) 374, obsd (M+H) 375.

### Example 8

### 1-(2-Isopropyl-phenyl)-3-(3-oxo-1-propyl-2,3-dihydro-1H-indazol-5-yl)-urea

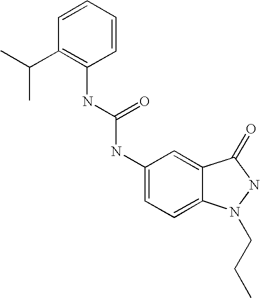

Following the general method 1, described above, 1-(2-isopropyl-phenyl)-3-(3-oxo-1-propyl-2,3-dihydro-1H-indazol-5-yl)-urea was prepared from 1-allyl-5-nitro-1,2-dihydro-indazol-3-one and 2-isopropylphenyl isocyanate (Yield: 83%). ES-MS calcd for C20H24N4O2 (m/e) 352, obsd 353 (M+H).

### Example 9

### 1-(3,4-Dichloro-phenyl)-3-(3-oxo-1-propyl-2,3-dihydro-1H-indazol-5-yl)-urea

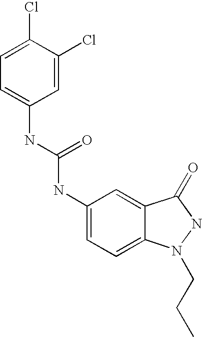

Following the general method 1, described above, 1-(3,4-dichloro-phenyl)-3-(3-oxo-1-propyl-2,3-dihydro-1H-indazol-5-yl)-urea was prepared from 1-allyl-5-nitro-1,2-dihydro-indazol-3-one and 3,4-dichlorophenyl isocyanate (Yield: 69%). ES-MS calcd for C17H16Cl2N4O2 (m/e) 378, obsd 379 (M+H).

### Example 10

### 1-(2-Ethyl-phenyl)-3-(3-oxo-1-propyl-2,3-dihydro-1H-inidazol-5-yl)-urea

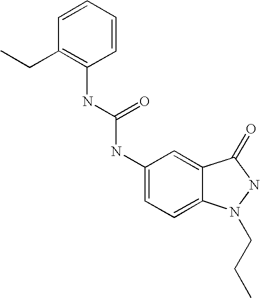

Following the general method 1, described above, 1-(2-ethyl-phenyl)-3-(3-oxo-1-propyl-2,3-dihydro-1H-indazol-5-yl)-urea was prepared from 1-allyl-5-nitro-1,2-dihydro-indazol-3-one and 2-ethylphenyl isocyanate (Yield; 83%). ES-MS calcd for C19H22N4O2 (m/e) 338, obsd 339 (M+H).

### Example 11

### 1-(2,5-Dichloro-phenyl)-3-(3-oxo-1-propyl-2,3-dihydro-1H-indazol-5-yl)-urea

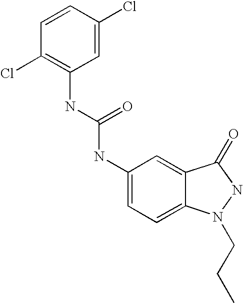

Following the general method 1, described above, 1-(2,5-dichloro-phenyl)-3-(3-oxo-1-propyl-2,3-dihydro-1H-indazol-5-yl)-urea was prepared from 1-allyl-5-nitro-1,2-dihydro-indazol-3-one and 2,5-dichlorophenyl isocyanate (Yield: 74%). ES-MS calcd for C17H16Cl2N4O2 (m/e) 378, obsd 379 (M+H).

### Example 12

### 1-(2,4-Dichloro-phenyl)-3-(3-oxo-1-propyl-2,3-dihydro-1H-indazol-5-yl)-urea

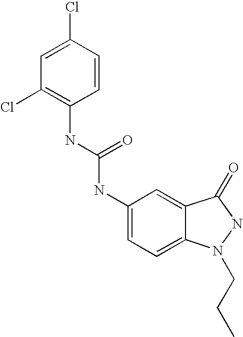

Following the general method 1, described above, 1-(2,4-dichloro-phenyl)-3-(3-oxo-1-propyl-2,3-dihydro-1H-indazol-5-yl)-urea was prepared from 1-allyl-5-nitro-1,2-dihydro-indazol-3-one and 2,4-dichlorophenyl isocyanate (Yield: 5 1 %). ES-MS calcd for C17H16C12N4O2 (m/e) 378, obsd 379 (M+H).

### Example 13

### 1-(3-Oxo-1-propyl-2,3-dihydro-1H-indazol-5-yl)-3-(4-trifluoromethyl-phenyl)-urea

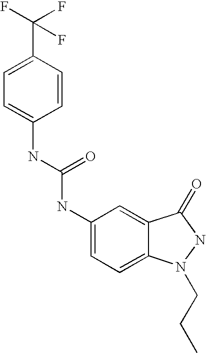

Following the general method 1, described above, 1-(3-oxo-1-propyl-2,3-dihydro-1 H-indazol-5-yl)-3-(4-trifluoromethyl-phenyl)-urea was prepared from 1-allyl-5-nitro-1,2-dihydro-indazol-3-one and 4-(trifluoromethyl)phenyl isocyanate (Yield: 79%). ES-MS calcd for C18H17F3N4O2 (m/e) 378, obsd 379 (M+H).

### Example 14

### 1-(3-Oxo-1-propyl-2,3-dihydro-1H-indazol-5-yl)-3-phenyl-urea

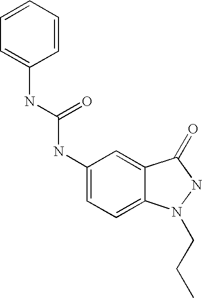

Following the general method 1, described above, 1-(3-oxo-1-propyl-2,3-dihydro-1H-indazol-5-yl)-3-phenyl-urea was prepared from 1-allyl-5-nitro-1,2-dihydro-indazol-3-one and phenyl isocyanate (Yield, 80%). ES-MS calcd for C17H18N4O2 (m/e) 310, obsd 311 (M+H).

### Example 15

### 1-(3-Chloro-phenyl)-3-(3-oxo-1-propyl-2,3-dihydro-1H-indazol-5-yl)-urea

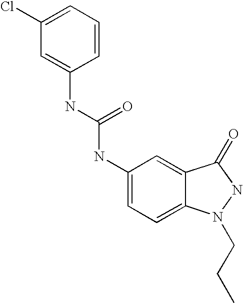

Following the general method 1, described above, 1-(3-chloro-phenyl)-3-(3-oxo-1-propyl-2,3-dihydro-1H-indazol-5-yl)-urea was prepared from 1-allyl-5-nitro-1,2-dihydro-indazol-3-one and 3-chlorophenyl isocyanate (Yield: 80%). ES-MS calcd for C17H17C1N4O2 (m/e) 344, obsd 345 (M+H).

### Example 16

### 1-(3-Oxo-1-propyl-2,3-dihydro-1H-indazol-5-yl)-3-m-tolyl-urea

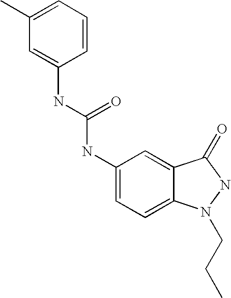

Following the general method 17 described above, 1-(3-oxo-1-propyl-2,3-dihydro-1H-indazol-5-yl)-3-m-tolyl-urea was prepared from 1-allyl-5-nitro-1,2-dihydro-indazol-3-one and m-tolyl isocyanate (Yield 80%). ES-MS calcd for C18H20N4O2 (m/e) 324, obsd 325 (M+H).

### Example 17

### 1-(3-Fluoro-phenyl)-3-(3-oxo-1-propyl-2,3-dihydro-1H-indazol-5-yl)-urea

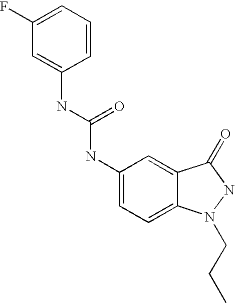

Following the general method 1, described above, 1-(3-fluoro-phenyl)-3-(3-oxo-1-propyl-2,3-dihydro-1H-indazol-5-yl)-urea was prepared from 1-allyl-5-nitro-1,2-dihydro-indazol-3-one and 3-fluoro-phenyl isocyanate (Yield: 76%). ES-MS calcd for Cl7H17FN4O2 (m/e) 328, obsd 329 (M+H).

### Example 18

### 1-(2,4-Difluoro-phenyl)-3-(3-oxo-1-propyl-2,3-dihydro-1H-indazol-5-yl)-urea

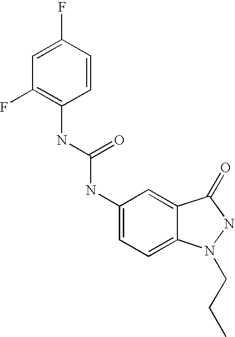

Following the general method 15 described above, 1-(2,4-difluoro-phenyl)-3-(3-oxo-1-propyl-2,3-dihydro-1H-indazol-5-yl)-urea was prepared from 1-allyl-5-nitro-1,2-dihydro-indazol-3-one and 2,4-difluorophenyl isocyanate (Yield: 77%). ES-MS calcd for C17H16F2N4O2 (m/e) 346, obsd 347 (M+H).

### Example 19

### 1-(3-Bromo-phenyl)-3-(3-oxo-1-propyl-2,3-dihydro-1H-indazol-5-yl)-urea

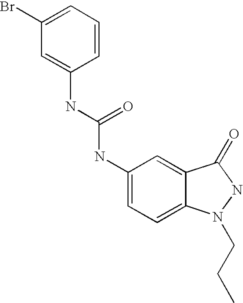

Following the general method 1, described above, 1-(3-bromo-phenyl)-3-(3-oxo-1-propyl-2,3-dihydro-1H-indazol-5-yl)-uea was prepared from 1-allyl-5-nitro-1,2-dihydro-indazol-3-one and 3-bromophenyl isocyanate (Yield: 85%). ES-MS calcd for C17H17BrN4O2 (m/e) 389, obsd 390 (M+H).

### Example 20

### 1-(2-Methoxy-phenyl)-3-(3-oxo-1-propyl-2,3-dihydro-1H-indazol-5-yl)-urea

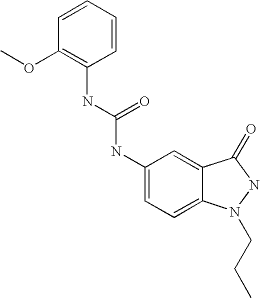

Following the general method 1, described above, 1-(2-methoxy-phenyl)-3-(3-oxo-1-propyl-2,3-dihydro-1H-indazol-5-yl)-utrea was prepared from 1-allyl-5-nitro-1,2-dihydro-indazol-3-one and 2-methoxyphenyl isocyanate (Yield: 74%). ES-MS calcd for C18H20N4O3 (m/e) 340, obsd 341 (M+H).

### Example 21

### 1-(2-Chloro-phenyl)-3-(3-oxo-1-propyl-2,3-dihydro-1H-indazol-5-yl)-urea

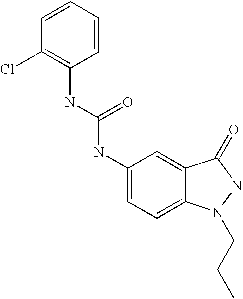

Following he general method 1, described above, 1-(2-chloro-phenyl)-3-(3-oxo-1-propyl-2,3-dihydro-1H-indazol-5-yl)-urea was prepared from 1-allyl-5-nitro-1,2-dihydro-indazol-3-one and 2-chlorophenyl isocyanate (Yield: 82%). ES-MS calcd for C17H17ClN4O2 (m/e) 344, obsd 345(M+H).

### Example 22

### 1-(2-Fluoro-phenyl)-3-(3-oxo-1-propyl-2,3-dihydro-1H-indazol-5-yl)-urea

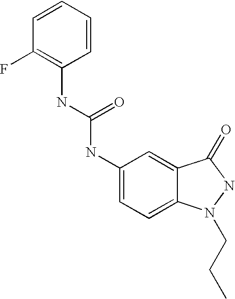

Following the general method 1, described above, 1-(2-fluoro-phenyl)-3-(3-oxo-1-propyl-2,3-dihydro-1H-indazol-5-yl)-urea was prepared from 1-allyl-5-nitro-1,2-dihydro-indazol-3-one and 2-fluorophenyl isocyanate (Yield:7? %), ES-MS calcd for Cl7H17FN4O2 (m/e) 328, obsd 329 (M+H).

### Example 23

### 1-(1-Benzyl-3-oxo-2,3-dihydro-1H-indazol-5-yl)-3-(3-chloro-2-methoxy-phenyl)-urea

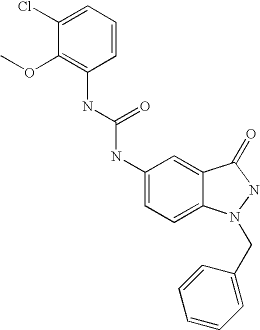

Following the general method 1, described above, 1-(1-benzyl-3-oxo-2,3-dihydro-1H-indazol-5-yl)-3-(3-chloro-2-methoxy-phenyl)-urea was prepared from 1-benzyl-5-nitro-1,2-dihydro-indazol-3-one and 3-chloro-2-methoxyphenyl isocyanate (Yield: 3%). ES-MS calcd for C22H19ClN4O3 (m/e) 422, obsd 423 (M+H).

### Example 24

### {5-[3-(2-ethyl-phenyl)-ureido]-3-oxo-2,3-dihydro-indazol-1-yl}-acetic acid ethyl ester

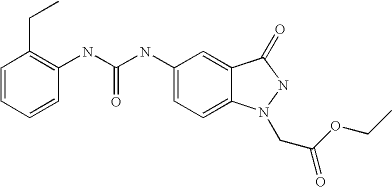

(5-Nitro-3-oxo-2,3-dihydro-indazol-1-yl)-acetic acid ethyl ester (201.1 mg, 0.758 mmol) in 10 ml ethanol in the presence of 39 mg 10% Pd/C was hydrogenated for I hr under 20 psi hydrogen. The reaction mixture was filtered through a Celite plug and evaporated to a light brown solid. The residue was dissolved in 5 ml dioxane under Ar and to this was added 2-ethylphenyl isocyanate (160 μL mg, 1.137 mmol). The mixture was heated to reflux for 1.5 hrs and then cooled. Water and ethyl acetate were added to the mixture and the organic layer was separated. The ethyl acetate solution was extracted with saturated sodium bicarbonate, dried over MgSO₄, filtered and evaporated to dryness. The residue was suspended in 30 mL H₂O and 8 mL ethyl acetate and the solid material was filtered and dried to yield the product {5-[3-(2-ethyl-phenyl)-ureido]-3-oxo-2,3-dihydro-indazol-1-yl}-acetic acid ethyl ester (23.2 mg, Yield: 36%). ES-MS calcd for C20H22N4O4 (m/e) 382 obsd 381 (M−H).

### Example 25

### 1-(2-ethyl-phenyl)-3-{1-[2-(2-methoxy-ethoxy)-ethyl]-3-oxo-2,3-dihydro-1H-indazol-5-yl}-urea

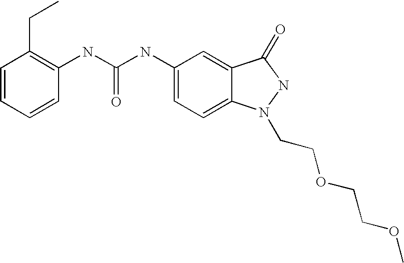

1-[2-(2-Methoxy-ethoxy)-ethyl]-5-nitro-1,2-dihydro-indazol-3-one (102.7 mg, 0.365 mmol) in 5 ml ethanol and 0.5 mL acetic acid in the presence of 30 mg 10% Pd/C was hydrogenated for 1 hr under 20 psi hydrogen, The reaction mixture was filtered through a Celite X plug, evaporated under vacuum, and re-evaporated from toluene to a purple oil. The residue was dissolved in 3 ml dioxane under Ar and to this was added 2-ethylphenyl isocyanate (62 FL mg, 0.438 mmol). The mixture was heated to reflux for 2 hrs and then cooled. Ethyl acetate and water were added to the mixture and the pH was adjusted to 6 using a pH 6 phosphate buffer. The aqueous solution was extracted with ethyl acetate. The combined organic layer was dried over MgSO₄, filtered and evaporated to dryness. The residue was purified by flash chromatography to yield 1-(2-ethyl-phenyl)-3-{1-[2-(2-methoxy-ethoxy)-ethyl]-3-oxo-2,3-dihydro-1H-indazol-5-yl}-urea (36 mg, Yield: 25%). ES-MS calcd for C21H26N4O4 (m/e) 398.46, obsd 397.2 (M−H).

### Example 26

### 1-(2-ethyl-phenyl)-3-[1-(2-methoxy-ethyl)-3-oxo-2,3-dihydro-1H-indazol-5-yl]-urea

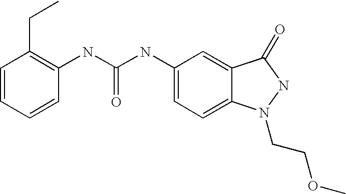

1-(2-Methoxy-ethyl)-5-nitro-1,2-dihydro-indazol-3-one (100 mg, 0.42 mmol) in 30 ml ethanol and 0.5 mL acetic acid in the presence of 30 mg 10% Pd/C was hydrogenated for 2 hrs under 20 psi hydrogen. The reaction mixture was filtered through a Celite® plug, evaporated under vacuum, and re-evaporated from toluene to a purple solid. The residue was dissolved in 3 ml dioxane under Ar and to this was added 2-ethylphenyl isocyanate (62 μL mg, 0.438 mmol). The mixture was heated to reflux for 1 h and then cooled. Ethyl acetate and 2.5% at. potassium bisulfate solution were added to the mixture and the precipitate was filtered off. The organic layer was washed with 2.5% potassium bisulfate solution, water, and saturated sodium-n chloride. The organic layer was dried over MgSO₄, filtered and evaporated to dryness. The residue was purified by flash chromatography to yield the product 1-(2-ethyl-phenyl)-3-[1-(2-methoxy-ethyl)-3-oxo-2,3-dihydro-1H-indazol-5-yl]-urea (66 mg, Yield: 45%). ES-MS calcd for C19H22N4O3 (m/e) 354.4, obsd 353.3 (M−H).

### General method for the preparation of 1-benzyl and 1-alkyl indazolone ureas from phosgene and an amine (General method 2)

A suspension of 1-benzyl-5-nitro-1.2-dihydro-indazol-3-one or 1-allyl-5-nitro-1,2-dihydro-indazol-3-one (1 eq.) and Pd/C (10%, 3-5% eq.) in MeOH (25 ml per 1mmol of substrate) was stirred under hydrogen atmosphere (balloon) at room temperature until completion of reduction. After removal of the catalyst and the solvent, the residue was dissolved in acetonitrile (5-15 ml) and evaporated again. The intermediate was then dried in high vacuum and then dissolved in dry THF (0.1 ml/mmol) and was added dropwise to a cold solution of phosgene in toluene (20%, 4.3 equiv.). After stirring at room temperature for 30 min, excess phosgene and solvents were removed in vacuo. The residue was then diluted with THF to be a 0.1 M solution. The solution was dispensed to vials (1 ml of solution in each vial) containing appropriate amines (40-60 mg). Then followed addition of neat triethylamine (0.2 ml) and the vials were shaken at 85 ° C. for 3 hr. The vials were then cooled and water (5 ml) was added and the mixtures were extracted with ethyl acetate. Liquid-liquid handling was carried out on Tecan. After removal of solvents, the residues were purified by HPLC to yield the pure products.

### Example 27

### 1-(3-Oxo-1-propyl-2,3-dihydro-1H-indazol-5-yl)-3-quinolin-8-yl-urea

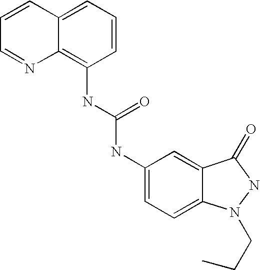

Following the general method 2, described above, 1-(3-oxo-1-propyl-2,3-dihydro-1H-indazol-5-yl)-3-quinolin-8-yl-urea was prepared from 1-allyl-5-nitro-1,2-dihydro-indazol-3-one and 8-aminoquinoline (Yield: 38%). ES-MS calcd for C2019N5O2 (m/e) 361, obsd 362 (M+H).

### Example 28

### 1-(3-Oxo-1-propyl-2,3-dihydro-1H-indazol-5-yl)-3-(2-piperidin-1-yl-phenyl)-urea

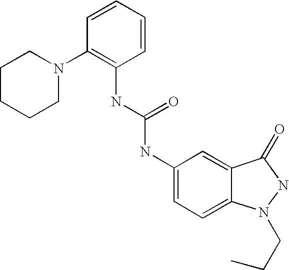

Following the general method 2, described above, 1-(3-oxo-1-propyl-2,3-dihydro-1H-indazol-5-yl)-3-(2-piperidin-1-yl-phenyl)-urea was prepared from 1-allyl-5-nitro-1,2-dihydro-indazol-3-one and 2-piperidinoaniline (Yield: 15%). ES-MS calcd for C22H27N502 (m/e) 393, obsd 394 (M−H).

### Example 29

### 1-(2-sec-Butyl-phenyl)-3-(3-oxo-1-propyl-2,3-dihydro-1H-indazol-5-yl)-urea

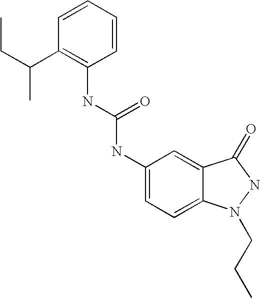

Following the general method 2, described above, 1-(2-sec-Butyl-phenyl)-3-(3-oxo-1-propyl-2,3-dihydro-1H-indazol-5-yl)-urea was prepared from 1-allyl-5-nitro-1,2-dihydro-indazol-3-one and 2-sec-butylanailine (Yield: 15%). ES-MS calcd for C21H26N4O2 (m/e) 366, obsd 367 (M+H).

### Example 30

### 1-(1-Benzyl-3-oxo-2,3-dihydro-1H-indazol-5-yl)-3-(2-piperidin-1-yl-phenyl)-urea

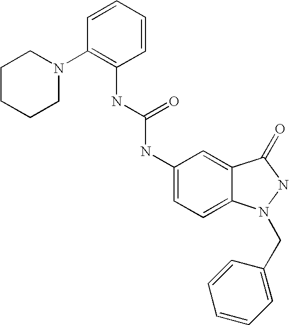

Following the general method 2, described above, 1-(1-Benzyl-3-oxo-2,3-dihydro-1H-indazol-5-yl)-3-(2-piperidin-1-yl-phenyl)-urea was prepared from 1-benzyl-5-nitro-1,2-dihydro-indazol-3-one and 2-piperidinoaniline. (Yield, 54%) ES-MS calcd for C26H27N5O2 (m/e) 441, obsd 442 (M+N).

### Example 31

### 1-(3-Ethynyl-phenyl)-3-(3-oxo-1-propyl-2,3-dihydro-1H-indazol-5-yl)-urea

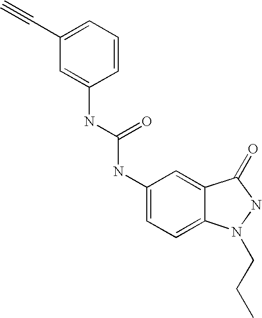

Following the general method 2, described above, 1-(3-ethynyl-phenyl)-3-(3-oxo-1-propyl-2,3-dihydro-1H-indazol-5-yl)-urea was prepared from 1-allyl-5-nitro-1,2-dihydro-indazol-3-one and 3-ethynylaniline (Yield: 34%). ES-MS calcd for C19H18N4O2 (m/e) 334, obsd 335 (M+H).

### Example 32

### 1-(3-Oxo-1-propyl-2,3-dihydro-1H-indazol-5-yl)-3-(3-trifluoromethyl-phenyl)-urea

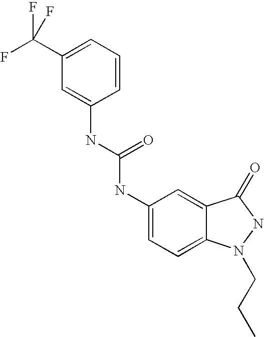

Following the general method 2, described above, 1-(3-oxo-1-propyl-2,3-dihydro-1H-indazol-5-yl)-3-(3-trifluoromethyl-phenyl)-urea was prepared from 1-allyl-5-nitro-1,2-dihydro-indazol-3-one and 3-trifluoromethyl aniline (Yield. 34%). ES-MS calcd for C18H17F3N4O2 (m/e) 378, obsd 379 (M+H).

### Example 33

### 1-(1-Benzyl-3-oxo-2,3-dihydro-1H-indazol-5-yl)-3-(3-isopropoxy-phenyl)-urea

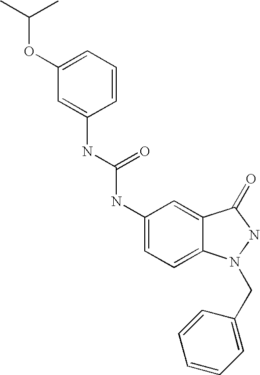

Following the general method 2, described above, 1-(1-benzyl-3-oxo-2,3-dihydro-1H-indazol-5-yl)-3-(3-isopropoxy-phenyl)-urea was prepared from 1-benzyl-5-nitro-1,2-dihydro-indazol-3-one and 3-isopropoxy aniline (Yield: 47%). ES-MS calcd for C24H24N4O3 (m/e) 416, obsd 417 (M+H).

### Example 34

### 1-(1-Benzyl-3-oxo-2,3-dihydro-1H-indazol-5-yl)-3-(3-trifluoromethyl-phenyl)-urea

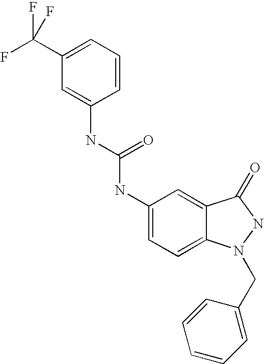

Following the general method 2, described above, 1-(1-benzyl-3-oxo-2,3-dihydro-1H-indazol-5-yl)-3-(3-trifluoromethyl-phenyl)-urea was prepared from 1-benzyl-5-nitro-1,2-dihydro-indazol-3-one and 3-trifluoromethyl aniline. (Yield: 43%). ES-MS calcd for C22H17F3N4O2 (m/e) 426, obsd 427 (M+H).

### Example 35

### DGAT Phospholipid FlashPlate Assay

Materials for the assay were: PL-FlashPlate: Phospholipid FlashPlates from PerkinElmer, catalog number SMP108; DAG (1,2-Dioleoyl-sn-glycerol) 10 mM suspended in water containing 0.1% Triton X-100; ¹⁴C-Pal-CoA (palmitoyl coenzyme A, [palmitoyl-1-¹⁴C]) from PerkinElmer, catalog number NEC-555 with a specific activity of 55 mCi/mmol; and DGAT pellet, with a protein concentration of 9.85 mg/ml.

Aqueous buffers were prepared or purchased as follows: The coating buffer (CB) was purchased from PerkinElmer, catalog number SMP900A; the reaction buffer (RB) was 50 mM Tris-HCl, pH 7.5, 100 mM NaCl, 0.01 % BSA in water; the washing buffer (WB) is 50 mM Tris-HCl, pH 7.5, 100 mM NaCl, 0.05 % deoxycholic acid sodium salt in water; the dilution buffer (DB) was 50 mM Tris-HCl, pH 7.5, 100 mM NaCl, 1 mM EDTA, 0.2% Triton X-100 in water.

1,2-Dioleoyl-sn-glycerol (DAG, 10 mmoles) was diluted to 500 μM with coating buffer (CB). The diluted DAG solution was then added to 384-well PL-FlashPlates at 60 μl per well, and incubated at room temperature for 2 days. The coated plates were then washed twice with washing buffer (WB) before use. Test compounds were serial diluted to 2000, 666.7, 222.2, 74.1, 24.7, 8.2, 2.7 and 0.9 μM in 100% DMSO. Diluted compound were further diluted 10 fold with reaction buffer (RB). ¹⁴C-Pal-CoA was diluted to 8.3 μM with RB. The DGAT pellet was diluted to 0.13 mg protein/ml with dilution buffer (DB) immediately before it was added to the PL-FlashPlates to start the reaction. 20 μl of the RB-diluted compounds (or 10% DMSO in RB for Total and Blank), 15 μl of RB diluted 14C-Pal-CoA and 15 μl of DB diluted DGAT pellet (DB without DGAT for Blanks) were transferred to each well of the PL-FlashPlates. The reaction mixtures were incubated at 37° C. for 1 hour. The reactions were stopped by washing 3 times with WB. Plates were sealed with Top-seal and read on a Topcount instrument.

Calculation of IC₅₀: The IC₅₀ values for each compound were generated using an Excel template. The Topcount rpm readings of Total and Blank were used as 0% and 100% inhibition. The percent inhibition values of reactions in the presence of compounds were calculated, and plotted against compound concentrations. All data were fitted into a Dose Response One Site model (4 parameter logistic model) as the following:

(A+((B−A)/(1+((x/C)̂D)))),

with A and B as the bottom and top of the curve (highest and lowest inhibition), respectively, and C as IC₅₀ and D as Hill Coefficient of the compound. The results are summarized in Table 1 below:

TABLE 1

| | Activity in DGAT Phospholipid |
|---|---|
| | FlashPlate Assay |
| | (A = IC₅₀ < 0.10 μM, |
| Compound | B = IC₅₀ ≧ 0.10 μM) |
| Example 1 | B |
| Example 2 | B |
| Example 3 | B |
| Example 4 | B |
| Example 5 | B |
| Example 6 | B |
| Example 7 | A |
| Example 8 | B |
| Example 9 | A |
| Example 10 | A |
| Example 11 | B |
| Example 12 | B |
| Example 13 | B |
| Example 14 | B |
| Example 15 | B |
| Example 16 | B |
| Example 17 | B |
| Example 18 | B |
| Example 19 | B |
| Example 20 | B |
| Example 21 | B |
| Example 22 | B |
| Example 23 | B |
| Example 24 | B |
| Example 25 | B |
| Example 26 | B |
| Example 27 | A |
| Example 28 | B |
| Example 29 | B |
| Example 30 | B |
| Example 31 | B |
| Example 32 | B |
| Example 33 | B |
| Example 34 | B |

It is to be understood that the invention is not limited to the particular embodiments of the invention described above, as variations of the particular embodiments may be made and still fall within the scope of the appended claims.

What is claimed is:

1. A compound of formula (I): 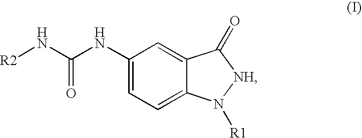
wherein:
R¹ is
—CH₂-aryl,
-lower alkyl,
—CH₂CH(O)OCH₂CH₃,
-alkoxy,
-alkoxy-alkoxy or
-alkoxy-lower alkyl; and
R² is
-aryl, unsubstituted or mono- or bisubstituted with halogen, lower alkyl, alkoxy, O-haloalkyl, haloalkyl, heterocycloalkyl or ethynyl moiety, or
-bicyclic heteroaryl;
and pharmaceutically acceptable salts thereof.

2. The compound according to claim 1, wherein R¹ is —CH₂-aryl and R² is aryl.

3. The compound according to claim 1, wherein R¹ is —CH₂-aryl and R₂ is bicyclic heteroaryl.

4. The compound according to claim 1, wherein R¹ is lower alkyl and R² is aryl.

5. The compound according to claim 1, wherein R¹ is lower alkyl and R² is bicyclic heteroaryl.

6. The compound according to claim 1, wherein R¹ is —CH₂-phenyl.

7. The compound according to claim 1, wherein R¹ is methyl, ethyl, propyl or butyl.

8. The compound according to claim 1, wherein R¹ is propyl.

9. The compound according to claim 1, wherein R¹ is —CH₂CH(O)OCH₂CH₃, —CH₂CH₂OCH₂CH₂OCH₃ or —CH₂CH₂OCH₃.

10. The compound according to claim 1, wherein R² is phenyl.

11. The compound according to claim 1, wherein R² is phenyl mono- or bisubstituted with a chlorine, bromine, fluorine, methoxy, ethoxy, trifluoromethoxy, methyl, ethyl, propyl, butyl, sec-butyl, isopropyl, trifluoromethyl, piperidine, ethynyl or isopropoxy moiety.

12. The compound according to claim 1, wherein R² is quinoline.

13. The compound according to claim 1, wherein said compound is:
1-(1-Benzyl-3-oxo-2,3-dihydro-1H-indazol-5-yl)-3-(2-ethoxy-phenyl)-urea,
1-(3-Oxo-1-propyl-2,3-dihydro-1H-indazol-5-yl)-3-(2-trifluoromethoxy-phenyl)-urea,
1-(5-Chloro-2-methoxy-phenyl)-3-(3-oxo-1-propyl-2,3-dihydro-1H-indazol-5-yl)-urea,
1-(3,4-Dichloro-phenyl)-3-(3-oxo-1-propyl-2,3-dihydro-1H-indazol-5-yl)-urea,
1-(2-Ethyl-phenyl)-3-(3-oxo-1-propyl-2,3-dihydro-1H-indazol-5-yl)-urea,
1-(3-Bromo-phenyl)-3-(3-oxo-1-propyl-2,3-dihydro-1H-indazol-5-yl)-urea,
1-(3-Oxo-1-propyl-2,3-dihydro-1H-indazol-5-yl)-3-quinolin-8-yl-urea,
1-(3-Oxo-1-propyl-2,3-dihydro-1H-indazol-5-yl)-3-(2-piperidin-1-yl-phenyl)-urea,
1-(2-sec-Butyl-phenyl)-3-(3-oxo-1-propyl-2,3-dihydro-1H-indazol-5-yl)-urea, and
1-(1-Benzyl-3-oxo-2,3-dihydro-1H-indazol-5-yl)-3-(2-ethoxy-phenyl)-urea.

14. A pharmaceutical composition, comprising a therapeutically effective amount of a compound according to claim 1 or a pharmaceutically acceptable salt thereof and a pharmaceutically acceptable carrier.
